(19) United States
(12) Patent Application Publication
(10) Pub. No.: US 2004/0116658 A1
(43) Pub. Date: Jun. 17, 2004

(54) Human KAP/Cdi1-related gene variant associated with small cell lung cancer

(76) Inventor: Ken-Shwo Dai, Hsinchu (TW)

Correspondence Address: Ladas & Parry, 26 West 61st Street, New York, NY, 10023, US

(21) Appl. No.: 10/103,957

(22) Filed: Mar. 22, 2002

(51) Int. Cl. C12Q 1/68; G01N 33/574; C07H 21/04; C07K 14/74; C07K 16/30; C12P 21/02; C12N 5/06

(52) U.S. Cl. 530/350; 536/23.5; 435/6; 530/388.22; 435/7.23; 435/69.1; 435/320.1; 435/325

(57) ABSTRACT

The invention relates to the nucleic acid and polypeptide sequences of a novel human KAP/Cdi1-related gene variant (KAP1).

The invention also relates to the process for producing the polypeptide of the variant.

The invention further relates to the use of the nucleic acid and polypeptide of the gene variant in diagnosing diseases, in particular, lung cancer, e.g. small cell lung cancer.

### FIELD OF THE INVENTION

The invention relates to the nucleic acid of a novel human KAP/Cdi1-related gene variant and the polypeptide encoded thereby, the preparation process thereof, and uses of the same in diagnosing diseases, in particular, lung cancers, e.g. small cell lung cancer (SCLC).

### BACKGROUND OF THE INVENTION

Lung cancer is one of the major causes of cancer-related deaths in the world. There are two primary types of lung cancers: small cell lung cancer and non-small cell lung cancer (NSCLC) (Carney, (1992a) Curr. Opin. Oncol. 4: 292-8). Small cell lung cancer accounts for approximately 25% of lung cancer and spreads aggressively (Smyth et al. (1986) Q J Med. 61: 969-76; Carney, (1992b) Lancet 339: 843-6). Non-small cell lung cancer represents the majority (about 75%) of lung cancer and is further divided into three main subtypes: squamous cell carcinoma, adenocarcinoma, and large cell carcinoma (Ihde and Minna, (1991) Curr Probl Cancer 15: 105-54). In recent years, much progress has been made toward understanding the molecular and cellular biology of lung cancers. Many important contributions have been made by the identification of several key genetic factors associated with lung cancers. However, the treatments of lung cancers still mainly depend on surgery, chemotherapy, and radiotherapy. This is because the molecular mechanisms underlying the pathogenesis of lung cancers remain largely unclear.

A recent hypothesis suggested that lung cancer is caused by genetic mutations of at least 10 to 20 genes (Sethi, (1997) BMJ. 314: 652-655). Therefore, future strategies for the prevention and treatment of lung cancers will be focused on the elucidation of these genetic substrates, in particular, the genes associated phosphatases since several members of this family (e.g., protein phosphatase 2A, CDC25A/B, and PTEN) have been shown to be associated with cancers (Parsons, (1998) Curr Opin Oncol 10:88-91). For example, protein phosphatase 2A has been shown to interact with the simian virus 40-small-t antigen, which in turn affect cell proliferation (Pallas et al. (1990) Cell 60:167-76; Sontag et al. (1993) Cell 75:887-97; Mungre et al. (1994) J Virol 68:1675-81). CDC25A/B is suggested to be an oncogene associated with human head and neck, breast, ovarian, oesophageal colorectal cancers and non-Hodgkin's lymphomas (Galaktionov et al. (1995) Science 269:1575-7; Gasparotto et al. (1997) Cancer Res 57:2366-8; Broggini et al. (2000) Anticancer Res 20:4835-40; Cangi et al. (2000) J Clin Invest 106:753-61; Nishioka et al. (2001) Br J Cancer 85:412-21; Hernandez et al. (2000) Int J Cancer 89:148-52; Hernandez et al. (2001) Lab Invest 81:465-73). PTEN has been shown to be associated with human brain, breast, prostate cancers (Li et al. (1997) Science 275:1943-7; Whang et al. (1998) Proc Natl Acad Sci USA 95:5246-50), and melanoma (Robertson, (1998) Proc Natl Acad Sci USA 95:9418-23), and mice neoplasms in organs of endometrium, liver, prostate, gastrointestinal tract, thyroid, and thymus (Podsypanina et al. (1999) Proc Natl Acad Sci USA 96:1563-8). PTEN has been reported to function as a tumor suppressor (Furnari et al. (1997) Proc Natl Acad Sci USA 94:12479-84; Cheney et al. (1998) Cancer Res 58:2331-4) mediated by the regulation of adhesion kinase (Tamura et al. (1998) Science 280:1614-7; Tamura et al. (1999) Cancer Res 59:442-9) or phosphoinositide 3-kinase/Akt pathway (Wu et al. (1998) Proc Natl Acad Sci USA 95:15587-91).

KAP/Cdi1, a phosphatase, was isolated as an interacting partner of the cyclin-dependent kinases using an interaction screening approach (Gyuris et al. (1993) Cell 75:791-803; Hannon et al. (1994) Proc Natl Acad Sci USA 91:1731-5). Based on the expression of KAP/Cdi1 transcript at the G1/S transition and the ability of its translated protein in interacting with Cdk2, Cdk3 and cdc2, it is suggested that KAP/Cdi1 plays a role in cell cycle regulation (Gyuris et al. (1993) Cell 75:791-803; Hannon et al. (1994) Proc Natl Acad Sci USA 91:1731-5). Overexpression of KAP/Cdi1 has been found in breast and prostate cancers (Lee et al. (2000) Mol Cell Biol 20:1723-32). Recently, the finding of KAP/Cdi1 aberrant transcripts in hepatocellular carcinoma (Yeh et al. (2000) Cancer Res 60:4697-700) suggests that mutations of KAP/Cdi1 may also be found in cancers of other tissues. Chromosomal mapping of KAP/Cdi1 on 14q22 (Demetrick et al. (1995) Cytogenet Cell Genet 69:190-2), an area associated with loss of heterozygosity in human lung cancers (Abujiang et al. (1998) Oncogene 17:3029-33), further strengthens that KAP/Cdi1 and/or its aberrant transcripts may play a role in lung cancers.

### SUMMARY OF THE INVENTION

The present invention provides one KAP/Cdi1-related gene variant present in human lung tissues. The nucleotide sequence of this variant and polypeptide sequence encoded thereby can be used for the diagnosis of diseases associated with this gene variant, in particular, lung cancers, e.g. small cell lung cancer.

The invention further provides an expression vector and host cell for expressing the polypeptide of the invention.

The invention further provides a method for producing the polypeptide encoded by the variant of the invention.

The invention further provides an antibody specifically binding to the polypeptide of the invention.

The invention also provides methods for diagnosing diseases associated with the deficiency of KAP/Cdi1-related gene, in particular, lung cancers, e.g. small cell lung cancer.

### BRIEF DESCRIPTION OF THE DRAWINGS

FIGS. 1A-B show the nucleic acid sequence (SEQ ID NO: 1) and amino acid sequence (SEQ ID NO:2) of KAP1.

FIGS. 2A-D shows the nucleotide sequence alignment between the human KAP/Cdi1 gene and its related gene variant (KAP1).

FIG. 3 shows the amino acid sequence alignment between the human KAP/Cdi1 protein and its related gene variant (KAP1).

### DETAILED DESCRIPTION OF THE INVENTION

According to the present invention, all technical and scientific terms used have the same meanings as commonly understood by persons skilled in the art.

The term “antibody” used herein denotes intact molecules (a polypeptide or group of polypeptides) as well as fragments thereof, such as Fab, R(ab′)₂, and Fv fragments, which are capable of binding the epitopic determinant. Antibodies are produced by specialized B cells after stimulation by an antigen. Structurally, an antibody consists of four subunits including two heavy chains and two light chains. The internal surface shape and charge distribution of the antibody binding domain is complementary to the features of an antigen. Thus, the antibody can specifically act against the antigen in an immune response.

The term “base pair (bp)” used herein denotes nucleotides composed of a purine on one strand of DNA which can be hydrogen bonded to a pyrimidine on the other strand. Thymine (or uracil) and adenine residues are linked by two hydrogen bonds. Cytosine and guanine residues are linked by three hydrogen bonds.

The term “Basic Local Alignment Search Tool (BLAST; Altschul et al., (1997) Nucleic Acids Res. 25: 3389-3402)” used herein denotes programs for evaluation of homologies between a query sequence (amino or nucleic acid) and a test sequence as described by Altschul et al. (Nucleic Acids Res. 25: 3389-3402, 1997). Specific BLAST programs are described as follows:

(1) BLASTN compares a nucleotide query sequence with a nucleotide sequence database;

(2) BLASTP compares an amino acid query sequence with a protein sequence database;

(3) BLASTX compares the six-frame conceptual translation products of a query nucleotide sequence with a protein sequence database;

(4) TBLASTN compares a query protein sequence with a nucleotide sequence database translated in all six reading frames; and

(5) TBLASTX compares the six-frame translations of a nucleotide query sequence with the six-frame translations of a nucleotide sequence database.

The term “cDNA” used herein denotes nucleic acids synthesized from a mRNA template using reverse transcriptase.

The term “cDNA library” used herein denotes a library composed of complementary DNAs which are reverse-transcribed from mRNAs.

The term “complement” used herein denotes a polynucleotide sequence capable of forming base pairing with another polynucleotide sequence. For example, the sequence 5′-ATGGACTTACT-3′ binds to the complementary sequence 5′-AGTAAGTCCAT-3′.

The term “deletion” used herein denotes a removal of a portion of one or more amino acid residues/nucleotides from a gene.

The term “expressed sequence tags (ESTs)” used herein denotes short (200 to 500 base pairs) nucleotide sequences derived from either 5′ or 3′ end of a cDNA.

The term “expression vector” used herein denotes nucleic acid constructs which contain a cloning site for introducing the DNA into the vector, one or more selectable markers for selecting vectors containing the DNA, an origin of replication for replicating the vector whenever the host cell divides, a terminator sequence, a polyadenylation signal, and a suitable control sequence which can effectively express the DNA in a suitable host. The suitable control sequence may include promoter, enhancer and other regulatory sequences necessary for directing polymerases to transcribe the DNA.

The term “host cell” used herein denotes a cell which is used to receive, maintain, and allow the reproduction of an expression vector comprising DNA. Host cells are transformed or transfected with suitable vectors constructed using recombinant DNA methods. The recombinant DNA introduced with the vector is replicated whenever the cell divides.

The term “insertion” or “addition” used herein denotes the addition of a portion of one or more amino acid residues/nucleotides to a gene.

The term “in silico” used herein denotes a process of using computational methods (e.g., BLAST) to analyze DNA sequences.

The term “polymerase chain reaction (PCR) used herein denotes a method which increases the copy number of a nucleic acid sequence using a DNA polymerase and a set of primers (about 20 bp oligonucleotides complementary to each strand of DNA) under suitable conditions (successive rounds of primer annealing, strand elongation, and dissociation).

The term “protein” or “polypeptide” used herein denotes a sequence of amino acids in a specific order that can be encoded by a gene or by a recombinant DNA. It can also be chemically synthesized.

The term “nucleic acid sequence” or “polynucleotide” used herein denotes a sequence of nucleotide (guanine, cytosine, thymine or adenine) in a specific order that can be a natural or synthesized fragment of DNA or RNA. It may be single-stranded or double-stranded.

The term “reverse transcriptase-polymerase chain reaction (RT-PCR)” used herein denotes a process which transcribes mRNA to complementary DNA strand using reverse transcriptase followed by polymerase chain reaction to amplify the specific fragment of DNA sequences.

The term “transformation” used herein denotes a process describing the uptake, incorporation, and expression of exogenous DNA by prokaryotic host cells.

The term “transfection” used herein denotes a process describing the uptake, incorporation, and expression of exogenous DNA by eukaryotic host cells.

The term “variant” used herein denotes a fragment of sequence (nucleotide or amino acid) inserted or deleted by one or more nucleotides/amino acids.

According to the present invention, the polypeptides of the novel human KAP/Cdi1-related gene variant and the fragments thereof, and the nucleic acid sequence encoding the same are provided.

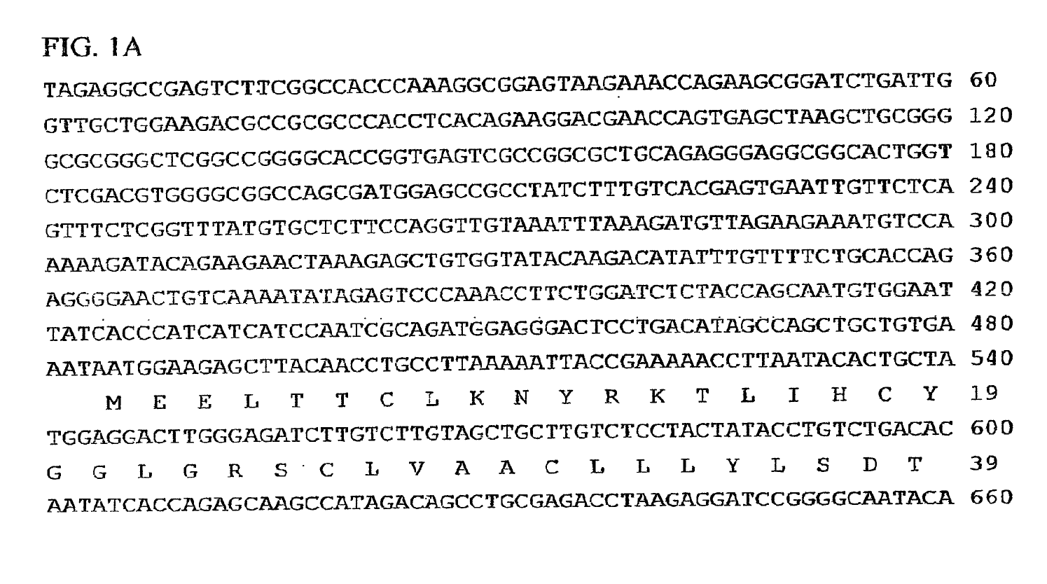

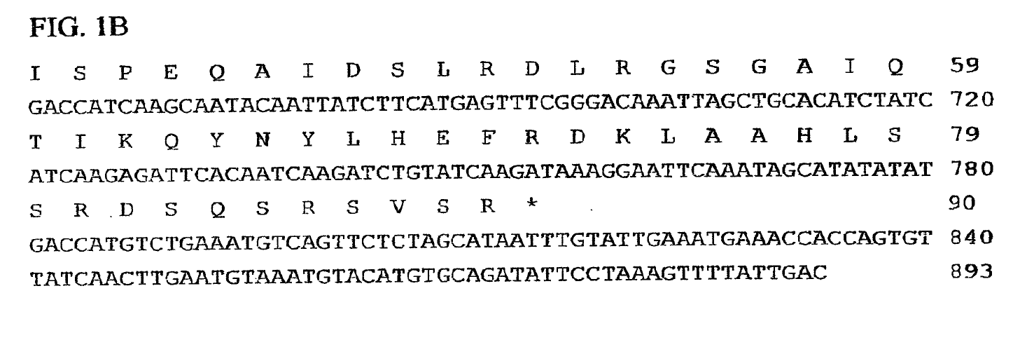

According to the present invention, human KAP/Cdi1 cDNA sequence was used to query the human lung EST databases (a normal lung and a small cell lung cancer) using BLAST program to search for KAP/Cdi1-related gene variants. One human cDNA partial sequence (i.e., EST) deposited in the databases showing similarity to KAP/Cdi1 was isolated and sequenced. This clones was isolated from small cell lung cancer cDNA library. FIGS. 1A-B shows the nucleic acid sequence of the variant (SEQ ID NO: 1) and its corresponding amino acid sequence encoded thereby (SEQ ID NO: 2). The full-length (893 bp) of the KAP1 cDNA clone contains an open reading frame (ORF) of 270 bp extending from nucleotides 485 to 754, which corresponds to an encoded protein of 90 amino acid residues with a predicted molecular mass of 10.2 kDa. The sequence around the initiation ATG codon of KAP1 (located at nucleotides 485 to 487) was similar to the Kozak consensus sequence (A/GCCATGG) (Kozak, (1987) Nucleic Acids Res. 15: 8125-48; Kozak, (1991) J Cell Biol. 115: 887-903.).

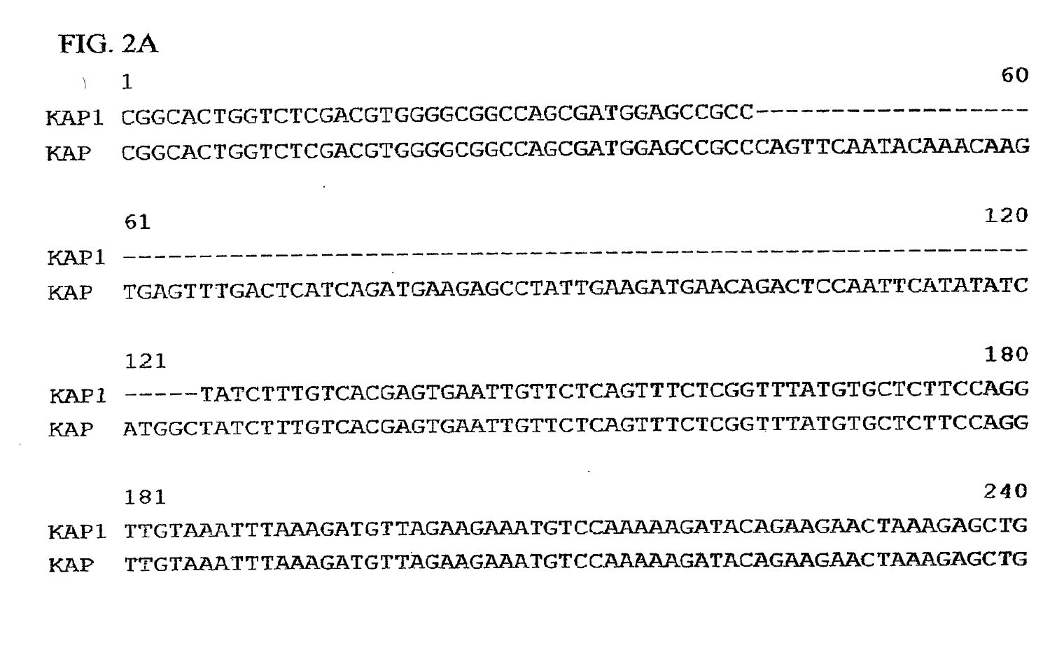

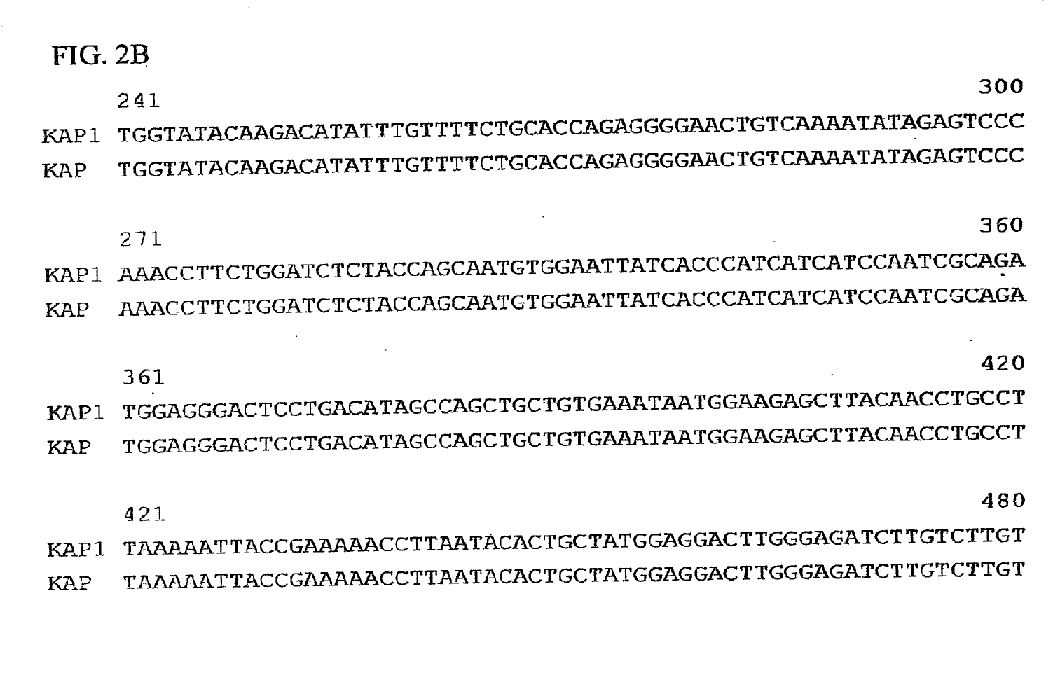

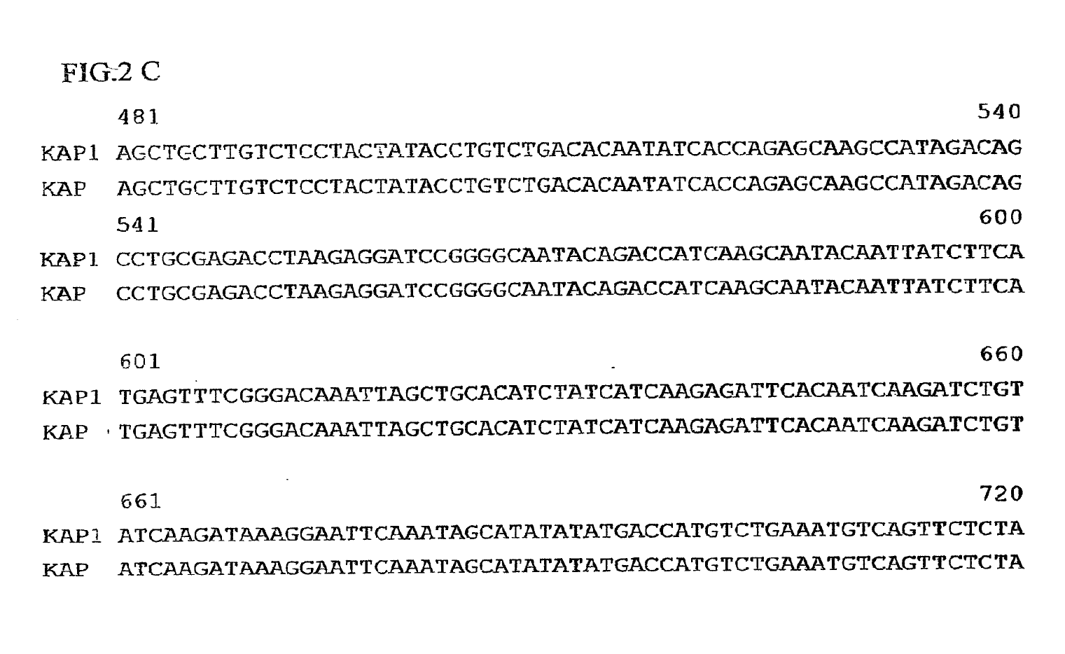

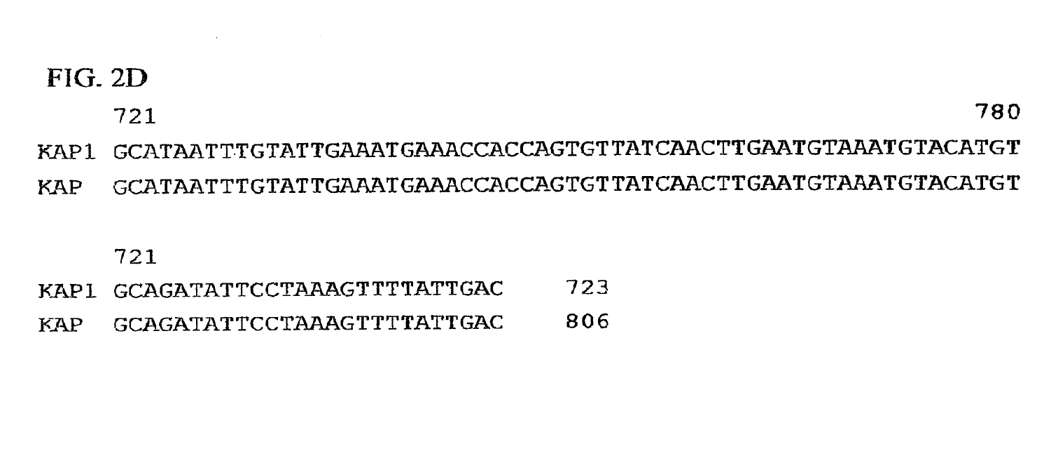

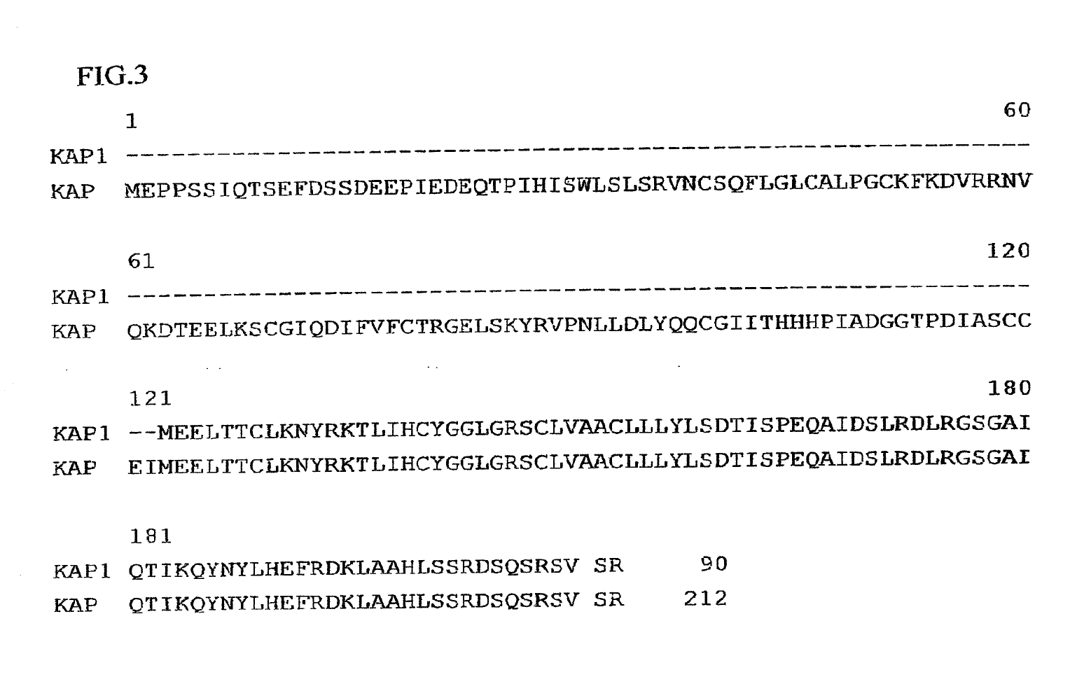

To determine whether KAP1 is a gene variant of KAP/Cdi1, alignments were performed to compare the nucleotide and amino acid sequences of KAP/Cdi1 and KAP1 (FIGS. 2A-D and 3). A major genetic alteration being found in the aligned nucleotide sequence shows that 83 bp nucleotides in the sequence of KAP/Cdi1 (nucleotides 43 to 125) are deleted from KAP1. This deletion eliminates the first ATG codon in the amino acid sequence found in KAP/Cdi, which results in the generation of an ORF starting from a downstream of the cloned KAP1 cDNA corresponding to the 123 amino acid residue of KAP/Cdi1. Thus, KAP1 encodes an N-terminally truncated gene variant of KAP/Cdi1 lacking the first 122 amino acids. Scanning of this 122 amino acid sequence against the profile entries in PROSITE (ScanProsite) indicated that this region contains one N-glycosylation site (nucleotides 38 to 41), and five Casein kinase II phosphorylation sites (nucleotides 10 to 13, 14 to 17, 15 to 18, 80 to 83, and 118 to 121).

The HCXXXXGR motif found in KAP/Cdi1 is an important functional motif serving as the catalytic core of protein tyrosine phosphatases (Charbonneau and Tonks, (1992) Annu Rev Cell Biol 8:463-93; Walton and Dixon, (1993) Annu Rev Biochem 62:101-20). This motif has been described in several tyrosine phosphatases. A comparison of the known phosphatases showed that no significant similarity in sequence could be found beyond the HCXXXXGR motif (Hannon et al. (1994) Proc Natl Acad Sci USA 91:1731-5; Barford et al. (1995) Nature Struct Biol 2:1043-1053; Denu et al. (1996) Cell 87:361-364). This important functional motif is also found in KAP1, which suggests that KAP1 is a novel member of the tyrosine phosphatase family.

Since a high degree of sequence variation beyond the motif region was found among the members of tyrosine phosphatase family, we believe that KAP1 may play a role which is specific to certain cell types, in particular, the cancer cells. To determine the distribution of KAP1 in different cancer cells, an in silico analysis was performed using a search of ESTs (originated from different cancer cell types) deposited in dbEST (Boguski et al., (1993) Nat Genet. 4: 332-3) at the National Center for Biotechnology Information (NCBI). Eight ESTs similar to KAP1 were identified for further analysis. Of these ESTs, two (GenBank accession #BE794067 and BE798063) showing the absence of 83 bp in their sequences were found to be originated from cDNA libraries generated using small cell lung carcinoma. The rest of six ESTs showing the presence of 83 bp in their sequences were originated from cDNA libraries generated using large cell lung carcinoma (GenBank accession #BE787225), germ cell tumors (GenBank accession #AI066521), colon tumor (GenBank accession #AI739135), glioblastoma (GenBank accession #BE738301), melanotic melanoma (GenBank accession #BG475390), and duodenal adenocarcinoma (GenBank accession #BG116357), respectively. This result suggests that the absence of the 83 bp nucleotide fragment located between nucleotides 242 and 243 of KAP1 is an important indicator for the small cell lung cancer. Therefore, any nucleotide fragments comprising nucleotides 240 to 245 of KAP1 may be used as probes for determining the presence of KAP1 under high stringency. An alternative approach is that any set of primers for amplifying the fragment containing nucleotide 240 to 245 of KAP1 may also be used for determining the presence of the variant.

According to the present invention, the polypeptides of the human KAP1 and fragments thereof may be produced through genetic engineering techniques. In this case, they are produced by appropriate host cells which have been transformed by DNAs that code for the polypeptides or the fragments thereof. The nucleotide sequence encoding the polypeptide of the human KAP1 or the fragments thereof is inserted into an appropriate expression vector, i.e., a vector which contains the necessary elements for the transcription and translation of the inserted coding sequence in a suitable host. The nucleic acid sequence is inserted into the vector in a manner that it will be expressed under appropriate conditions (e.g., in proper orientation and correct reading frame and with appropriate expression sequences, including an RNA polymerase binding sequence and a ribosomal binding sequence).

Any method that is known to those skilled in the art may be used to construct expression vectors containing a sequence encoding the polypeptide of the human KAP1 and appropriate transcriptional/translational control elements. These methods may include in vitro recombinant DNA and synthetic techniques, and in vivo genetic recombinants. (See, e.g., Sambrook, J. Cold Spring Harbor Press, Plainview N.Y., ch. 4, 8, and 16-17; Ausubel, R. M. et al. (1995) Current protocols in Molecular Biology, John Wiley & Sons, New York N.Y., ch. 9, 13, and 16.)

A variety of expression vector/host systems may be utilized to express the polypeptide-coding sequence. These include, but not limited to, microorganisms such as bacteria transformed with recombinant bacteriophage, plasmid, or cosmid DNA expression vector; yeast transformed with yeast expression vector; insect cell systems infected with virus (e.g., baculovirus); plant cell system transformed with viral expression vector (e.g., cauliflower mosaic virus, CaMV, or tobacco mosaic virus, TMV); or animal cell system infected with virus (e.g., vaccina virus, adenovirus, etc.). Preferably, the host cell is a bacterium, and most preferably, the bacterium is E. coli.

Alternatively, the polypeptides of the human KAP1 or the fragments thereof may be synthesized using chemical methods. For example, peptide synthesis can be performed using various solid-phase techniques (Roberge, J. Y. et al. (1995) Science 269: 202 to 204). Automated synthesis may be achieved using the ABI 431A peptide synthesizer (Perkin-Elmer).

According to the present invention, the fragments of the polypeptide and the nucleic acid sequence of the human KAP1 can be used as immunogens and primers or probes, respectively. Preferably, the purified fragments of the human KAP1 are used. The fragments may be produced by enzyme digestion, chemical cleavage of isolated or purified polypeptide or nucleic acid sequences, or chemical synthesis and then may be isolated or purified. Such isolated or purified fragments of the polypeptides and nucleic acid sequences can be directly used as immunogens and primers or probes, respectively.

The present invention further provides the antibodies which specifically bind one or more out-surface epitopes of the polypeptides of the human KAP1.

According to the present invention, immunization of mammals with immunogens described herein, preferably humans, rabbits, rats, mice, sheep, goats, cows, or horses, is performed following procedures well known to those skilled in the art, for the purpose of obtaining antisera containing polyclonal antibodies or hybridoma lines secreting monoclonal antibodies.

Monoclonal antibodies can be prepared by standard techniques, given the teachings contained herein. Such techniques are disclosed, for example, in U.S. Pat. No. 4,271,145 and 4,196,265. Briefly, an animal is immunized with the immunogen. Hybridomas are prepared by fusing spleen cells from the immunized animal with myeloma cells. The fusion products are screened for those producing antibodies that bind to the immunogen. The positive hybridoma clones are isolated, and the monoclonal antibodies are recovered from those clones.

Immunization regimens for production of both polyclonal and monoclonal antibodies are well-known in the art. The immunogen may be injected by any of a number of routes, including subcutaneous, intravenous, intraperitoneal, intradermal, intramuscular, mucosal, or a combination thereof. The immunogen may be injected in soluble form, aggregate form, attached to a physical carrier, or mixed with an adjuvant, using methods and materials well-known in the art. The antisera and antibodies may be purified using column chromatography methods well known to those skilled in the art.

According to the present invention, antibody fragments which contain specific binding sites for the polypeptides or fragments thereof may also be generated. For example, such fragments include, but are not limited to, F(ab′)₂ fragments produced by pepsin digestion of the antibody molecule and Fab fragments generated by reducing the disulfide bridges of the F(ab′)₂ fragments.

Many gene variants have been found to be associated with diseases (Stallings-Mann et al., (1996) Proc Natl Acad Sci USA 93: 12394-9; Liu et al., (1997) Nat Genet 16:328-9; Siffert et al., (1998) Nat Genet 18: 45 to 8; Lukas et al., (2001) Cancer Res 61: 3212 to 9). Since KAP1, a gene variant of KAP/Cdi1 (a tyrosine phosphatase associated with cell cycle regulation), is located on a region (chromosome 14q) of frequent loss of heterozygosity in lung cancer, it is advisable that the gene variant of the present invention, which has genetic deletion of nucleotide/amino acid sequences, may result in cancer development and be useful as markers for the diagnosis of human lung cancer. Based on the source of ESTs generated (cDNA libraries), the in silico tissue distribution analysis showed that KAP1 is associated with SCLC. Thus, the expression level of KAP1 relative to the expression level of KAP/Cdi1 may be a useful indicator for screening of patients suspected of having SCLC. This suggests that the index of relative expression level (mRNA or protein) may confer an increased susceptibility to SCLC. Fragment of KAP1 gene transcript (mRNA) may be detected by RT-PCR approach. Polypeptides of KAP1 may be determined by the binding of antibodies to these polypeptides. These approaches may be performed in accordance with conventional methods well known by persons skilled in the art.

The subject invention also provides methods for diagnosing the diseases associated with the deficiency of KAP/Cdi1 in a mammal, in particular, lung cancer, e.g. small cell lung cancer.

The method for diagnosing the diseases associated with the deficiency of KAP/Cdi1 may be performed by detecting the nucleotide sequence of KAP1 of the invention which comprises the steps of: (1) extracting total RNA of cells obtained from a mammal; (2) amplifying the RNA by reverse transcriptase-polymerase chain reaction (RT-PCR) with a set of primers to obtain a cDNA comprising the fragments comprising nucleotides 240 to 245 of SEQ ID NO: 1; and (3) detecting whether the cDNA sample is obtained. If necessary, the amount of the obtained cDNA sample may be detected.

In the above embodiment, one of the primers may be designed to have a sequence comprising the nucleotides of SEQ ID NO: 1 containing nucleotides 240 to 245, and the other may be designed to have a sequence complementary to the nucleotides of SEQ ID NO: 1 at any other locations downstream of nucleotide 245. Alternatively, one of the primers may be designed to have a sequence complementary to the nucleotides of SEQ ID NO: 1 containing nucleotides 240 to 245, and the other may be designed to have a sequence comprising the nucleotides of SEQ ID NO: 1 at any other locations upstream of nucleotide 240. In this case, only KAP1 will be amplified.

Alternatively, one of the primers may be designed to have a sequence comprising the nucleotides of SEQ ID NO: 1 upstream of nucleotide 242 and the other may be designed to have a sequence complementary to the nucleotides of SEQ ID NO: 1 downstream of nucleotide 243. Alternatively, one of the primers may be designed to have a sequence complementary to the nucleotides of SEQ ID NO: 1 upstream of nucleotide 242 and the other may be designed to have a sequence comprising the nucleotides of SEQ ID NO: 1 downstream of nucleotide 243. In this case, both KAP/Cdi1 and KAP1 will be amplified. The length of the PCR fragment from KAP1 will be 83 bp shorter than that from KAP/Cdi1.

Preferably, the primers of the invention contain 15 to 30 nucleotides.

Total RNA may be isolated from patient samples by using TRIZOL reagents (Life Technology). Tissue samples (e.g., biopsy samples) are powdered under liquid nitrogen before homogenization. RNA purity and integrity are assessed by absorbance at 260/280 nm and by agarose gel electrophoresis. The set of primers designed to amplify the expected sizes of specific PCR fragments of gene variant (KAP1) can be used. PCR fragments are analyzed on a 1% agarose gel using five microliters (10%) of the amplified products. To determine the expression levels for each gene variants, the intensity of the PCR products may be determined by using the Molecular Analyst program (version 1.4.1; Bio-Rad).

The RT-PCR experiment may be performed according to the manufacturer instructions (Boehringer Mannheim). A 50 μl reaction mixture containing 2 μl total RNA (0.1 μg/μl), 1 μl each primer (20 pM), 1 μl each dNTP (10 mM), 2.5 μl DTT solution (100 mM), 10 μl 5×RT-PCR buffer, 1 μl enzyme mixture, and 28.5 μl sterile distilled water may be subjected to the conditions such as reverse transcription at 60° C. for 30 minutes followed by 35 cycles of denaturation at 94° C. for 2 minutes, annealing at 60° C. for 2 minutes, and extension at 68° C. for 2 minutes. The RT-PCR analysis may be repeated twice to ensure reproducibility, for a total of three independent experiments.

Another embodiment of the method for diagnosing the diseases associated with the deficiency of KAP/Cdi1 may be performed by detecting the nucleotide sequences of KAP1 of the invention, which comprises the steps of: (1) extracting total RNA from a sample obtained from the mammal; (2) amplifying the RNA by reverse transcriptase-polymerase chain reaction (RT-PCR) to obtain a cDNA sample; (3) bringing the cDNA sample into contact with the nucleic acid of SEQ ID NO: 1 and the fragments thereof; and (4) detecting whether the cDNA sample hybridizes with the nucleic acid of SEQ ID NO: 1 or the fragments thereof. If necessary, the amount of hybridized sample may be detected.

The expression of gene variants can be analyzed using Northern Blot hybridization approach. Specific fragment comprising nucleotides 240 to 245 of the KAP1 may be amplified by polymerase chain reaction (PCR) using primer set designed for RT-PCR. The amplified PCR fragment may be labeled and serve as a probe to hybridize the membranes containing total RNAs extracted from the samples under the conditions of 55° C. in a suitable hybridization solution for 3 hours. Blots may be washed twice in 2×SSC, 0.1% SDS at room temperature for 15 minutes each, followed by two washes in 0.1×SSC and 0.1% SDS at 65° C. for 20 minutes each. After these washes, blot may be rinsed briefly in suitable washing buffer and incubated in blocking solution for 30 minutes, and then incubated in suitable antibody solution for 30 minutes. Blots may be washed in washing buffer for 30 minutes and equilibrated in suitable detection buffer before detecting the signals. Alternatively, the presence of gene variants (cDNAs or PCR) can be detected using microarray approach. The cDNAs or PCR products corresponding to the nucleotide sequences of the present invention may be immobilized on a suitable substrate such as a glass slide. Hybridization can be preformed using the labeled mRNAs extracted from samples. After hybridization, nonhybridized mRNAs are removed. The relative abundance of each labeled transcript, hybridizing to a cDNA/PCR product immobilized on the microarray, can be determined by analyzing the scanned images.

According to the present invention, the method for diagnosing the diseases associated with the deficiency of KAP/Cdi1 may also be performed by detecting the polypeptide of the gene variant of the invention. For instance, the polypeptide in protein samples obtained from the mammal may be determined by, but is not limited to, the immunoassay wherein the antibody specifically binding to the polypeptide of the invention is contacted with the protein samples, and the antibody-polypeptide complex is detected. If necessary, the amount of antibody-polypeptide complex can be determined.

The polypeptides of the gene variants may be expressed in prokaryotic cells by using suitable prokaryotic expression vectors. The cDNA fragments of KAP1 gene encoding the amino acid coding sequence may be PCR amplified using primer set with restriction enzyme digestion sites incorporated in the 5′ and 3′ ends, respectively. The PCR products can then be enzyme digested, purified, and inserted into the corresponding sites of prokaryotic expression vector in-frame to generate recombinant plasmids. Sequence fidelity of this recombinant DNA can be verified by sequencing. The prokaryotic recombinant plasmids may be transformed into host cells (e.g., E. coli BL21 (DE3)). Recombinant protein synthesis may be stimulated by the addition of 0.4 mM isopropylthiogalactoside (IPTG) for 3 hours. The bacterially-expressed proteins may be purified.

The polypeptide of the gene variant may be expressed in animal cells by using eukaryotic expression vectors. Cells may be maintained in Dulbecco's modified Eagle's medium (DMEM) supplemented with 10% fetal bovine serum (FBS; Gibco BRL) at 37° C. in a humidified 5% CO₂ atmosphere. Before transfection, the nucleotide sequence of the gene variant may be amplified with PCR primers containing restriction enzyme digestion sites and ligated into the corresponding sites of eukaryotic expression vector in-frame. Sequence fidelity of this recombinant DNA can be verified by sequencing. The cells may be plated in 12-well plates one day before transfection at a density of 5×10⁴ cells per well. Transfections may be carried out using Lipofectamine Plus transfection reagent according to the manufacturer's instructions (Gibco BRL). Three hours following transfection, medium containing the complexes may be replaced with fresh medium. Forty-eight hours after incubation, the cells may be scraped into lysis buffer (0.1 M Tris HCl, pH 8.0, 0.1% Triton X-100) for purification of expressed proteins. After these proteins are purified, monoclonal antibodies against these purified proteins (KAP1) may be generated using hybridoma technique according to the conventional methods (de StGroth and Scheidegger, (1980) J Immunol Methods 35:1-21; Cote et al. (1983) Proc Natl Acad Sci USA 80: 2026-30; and Kozbor et al. (1985) J Immunol Methods 81:31-42).

According to the present invention, the presence of the polypeptide of the gene variant in samples of squamous cell lung cancer may be determined by, but is not limited to, Western blot analysis. Proteins extracted from samples may be separated by SDS-PAGE and transferred to suitable membranes such as polyvinylidene difluoride (PVDF) in transfer buffer (25 mM Tris-HCl, pH 8.3, 192 mM glycine, 20% methanol) with a Trans-Blot apparatus for 1 hour at 100 V (e.g., Bio-Rad). The proteins can be immunoblotted with specific antibodies. For example, membrane blotted with extracted proteins may be blocked with suitable buffers such as 3% solution of BSA or 3% solution of nonfat milk powder in TBST buffer (10 mM Tris-HCl, pH 8.0, 150 mM NaCl, 0.1% Tween 20) and incubated with the monoclonal antibody directed against the polypeptide of gene variant. Unbound antibody is removed by washing with TBST for 5×1 minutes. Bound antibody may be detected using commercial ECL Western blotting detecting reagents.

The following examples are provided for illustration, but not for limiting the invention.

### EXAMPLES

### Analysis of Human Lung EST Databases

Expressed sequence tags (ESTs) generated from the large-scale PCR-based sequencing of the 5′-end of human lung (normal and large cell lung cancer) cDNA clones were compiled and served as EST databases. Sequence comparisons against the nonredundant nucleotide and protein databases were performed using BLASTN and BLASTX programs (Altschul et al., (1997) Nucleic Acids Res. 25: 3389-3402; Gish and States, (1993) Nat Genet 3:266-272), at NCBI with a significance cutoff of p<10⁻¹⁰. ESTs representing putative KAP/Cdi1 encoding gene were identified during the course of EST generation.

### Isolation of cDNA Clones

A cDNA clones exhibiting EST sequences similar to the KAP/Cdi1 gene was isolated from the small cell lung cancer cDNA library and named KAP1. The inserts of these clones were subsequently excised in vivo from the λZAP Express vector using the ExAssist/XLOLR helper phage system (Stratagene). Phagemid particles were excised by coinfecting XL1-BLUE MRF′ cells with ExAssist helper phage. The excised pBluescript phagemids were used to infect E. coli XLOLR cells, which lack the amber suppressor necessary for ExAssist phage replication. Infected XLOLR cells were selected using kanamycin resistance. Resultant colonies contained the double stranded phagemid vector with the cloned cDNA insert. A single colony was grown overnight in LB-kanamycin, and DNA was purified using a Qiagen plasmid purification kit.

### Full Length Nucleotide Sequencing and Database Comparisons

Phagemid DNA was sequenced using the Epicentre#SE9101LC SequiTherm EXCEL™II DNA Sequencing Kit for 4200S-2 Global NEW IR² DNA sequencing system (LI-COR). Using the primer-walking approach, full-length sequence was determined. Nucleotide and protein searches were performed using BLAST against the non-redundant database of NCBI.

### In Silico Tissue Distribution Analysis

The coding sequence for each cDNA clones was searched against the dbEST sequence database (Boguski et al., (1993) Nat Genet. 4: 332-3) using the BLAST algorithm at the NCBI website. ESTs derived from each tissue were used as a source of information for transcript tissue expression analysis. Tissue distribution for each isolated cDNA clone was determined by ESTs matching to that particular sequence variant with a significance cutoff of p<10⁻¹⁰.

### REFERENCES

Abujiang et al., Loss of heterozygosity (LOH) at 17q and 14q in human lung canc,ers. Oncogene 17:3029-33, (1998)

Altschul et al., Gapped BLAST and PSI-BLAST: a new generation of protein database search programs, Nucleic Acids Res, 25: 3389-3402, (1997).

Ausubel et al., Current protocols in Molecular Biology, John Wiley & Sons, New York N.Y., ch. 9, 13, and 16, (1995).

Barford et al., Protein tyrosine phosphatases take off. Nature Struct Biol 2:1043-1053, (1995).

Boguski et al., dbEST—database for “expressed sequence tags”. Nat Genet. 4: 332-3, (1993).

Broggini et al., Cell cycle-related phosphatases CDC25A and B expression correlates with survival in ovarian cancer patients. Anticancer Res 20:4835-40, (2000).

Cangi et al., Role of the Cdc25A phosphatase in human breast cancer. J Clin Invest 106:753-61, (2000)

Carney, D. N. The biology of lung cancer. Curr. Opin. Oncol. 4: 292-8, (1992a).

Carney, D. N. Biology of small-cell lung cancer. Lancet 339: 843-6, (1992b).

Charbonneau and Tonks, 1002 protein phosphatases? Annu Rev Cell Biol 8:463-93 (1992).

Cheney et al., Suppression of tumorigenicity of glioblastoma cells by adenovirus-mediated MMAC1/PTEN gene transfer. Cancer Res 58:2331-4 (1998).

Cote et al., Generation of human monoclonal antibodies reactive with cellular antigens, Proc Natl Acad Sci USA 80: 2026-30, (1983).

de StGroth and Scheidegger, Production of monoclonal antibodies: strategy and tactics. J Immunol Methods 35:1-21, (1980).

Demetrick et al., Chromosomal mapping of the genes for the human cell cycle proteins cyclin C (CCNC), cyclin E (CCNE), p21 (CDKN1) and KAP (CDKN3). Cytogenet Cell Genet 69:190-2, (1995).

Denu et al., Form and function in protein phosphorylation. Cell 87:361-364, (1996).

Furnari et al., Growth suppression of glioma cells by PTEN requires a functional phosphatase catalytic domain. Proc Natl Acad Sci USA 94:12479-84, (1997).

Galaktionov et al., CDC25 phosphatases as potential human oncogenes. Science 269:1575-7, (1995).

Gasparotto et al., Overexpression of CDC25A and CDC25B in head and neck cancers. Cancer Res 57:2366-8, (1997).

Gyuris et al., Cdi1, a human G1 and S phase protein phosphatase that associates with Cdk2. Cell 75:791-803, (1993).

Hannon et al., KAP: a dual specificity phosphatase that interacts with cyclin-dependent kinases. Proc Natl Acad Sci USA 91:1731-5, (1994).

Hernandez et al., Fernandez P L, Cardesa A, Campo E. Differential expression of cdc25 cell-cycle-activating phosphatases in human colorectal carcinoma. Lab Invest 81:465-73, (2001).

Hernandez et al., cdc25a and the splicing variant cdc25b2, but not cdc25B 1, -B3 or -C, are over-expressed in aggressive human non-Hodgkin's lymphomas. Int J Cancer 89:148-52, (2000).

Ihde and Minna. Non-small cell lung cancer. Part II: Treatment. Curr. Probl. Cancer 15: 105-54, (1991).

Kozak, An analysis of 5′-noncoding sequences from 699 vertebrate messenger RNAs. Nucleic Acids Res, 15: 8125-48, (1987).

Kozak, An analysis of vertebrate mRNA sequences: intimations of translational control, J Cell Biol, 115: 887-903, (1991).

Kozbor et al., Specific immunoglobulin production and enhanced tumorigenicity following ascites growth of human hybridomas. J Immunol Methods 81:31-42, (1985).

Lee et al., Overexpression of kinase-associated phosphatase (KAP) in breast and prostate cancer and inhibition of the transformed phenotype by antisense KAP expression. Mol Cell Biol 20:1723-32, (2000).

Li et al., PTEN, a putative protein tyrosine phosphatase gene mutated in human brain, breast, and prostate cancer. Science 275:1943-7, (1997).

Liu et al., Silent mutation induces exon skipping of fibrillin-1 gene in Marfan syndrome. Nat Genet 16:328-9, (1997).

Lukas et al., Alternative and aberrant messenger RNA splicing of the mdm2 oncogene in invasive breast cancer. Cancer Res 61:3212-9, (2001).

Mungre et al., Mutations which affect the inhibition of protein phosphatase 2A by simian virus 40 small-t antigen in vitro decrease viral transformation. J Virol 68:1675-81, (1994).

Nishioka et al., Clinical significance of CDC25A and CDC25B expression in squamous cell carcinomas of the oesophagus. Br J Cancer 85:412-21, (2001).

Pallas et al., Polyoma small and middle T antigens and SV40 small t antigen form stable complexes with protein phosphatase 2A. Cell 60:167-76, (1990).

Parsons R. Phosphatases and tumorigenesis. Curr Opin Oncol 1998, 10:88-91

Podsypanina et al., Mutation of Pten/Mmac1 in mice causes neoplasia in multiple organ systems. Proc Natl Acad Sci USA 96:1563-8, (1999).

Roberge et al., A strategy for a convergent synthesis of N-linked glycopeptides on a solid support. Science 269:202-4, (1995).

Robertson et al., In vitro loss of heterozygosity targets the PTEN/MMAC1 gene in melanoma. Proc Natl Acad Sci USA 95:9418-23, (1998).

Sambrook, J. Cold Spring Harbor Press, Plainview N.Y., ch. 4, 8, and 16-17.

Sethi, Science, medicine, and the future. Lung cancer, BMJ, 314: 652-655, (1997)

Siffert et al., Association of a human G-protein beta3 subunit variant with hypertension. Nat Genet, 18:45-8, (1998).

Smyth et al., The impact of chemotherapy on small cell carcinoma of the bronchus. Q J Med, 61: 969-76, (1986).

Sontag et al., The interaction of SV40 small tumor antigen with protein phosphatase 2A stimulates the map kinase pathway and induces cell proliferation. Cell, 75:887-97, (1993).

Stallings-Mann et al., Alternative splicing of exon 3 of the human growth hormone receptor is the result of an unusual genetic polymorphism. Proc Natl Acad Sci USA 93:12394-9, (1996).

Tamura et al., Inhibition of cell migration, spreading, and focal adhesions by tumor suppressor PTEN. Science, 280:1614-7, (1998).

Tamura et al., Tumor suppressor PTEN inhibition of cell invasion, migration, and growth: differential involvement of focal adhesion kinase and p130Cas. Cancer Res 59:442-9, (1999).

Walton and Dixon. Protein tyrosine phosphatases. Annu Rev Biochem 62:101-20, (1993).

Whang et al., Inactivation of the tumor suppressor PTEN/MMAC1 in advanced human prostate cancer through loss of expression. Proc Natl Acad Sci USA 95:5246-50, (1998).

Wu et al., The PTEN/MMAC1 tumor suppressor phosphatase functions as a negative regulator of the phosphoinositide 3-kinase/Akt pathway. Proc Natl Acad Sci USA 95:15587-91, (1998).

Yeh et al., Aberrant transcripts of the cyclin-dependent kinase-associated protein phosphatase in hepatocellular carcinoma. Cancer Res 60:4697-700, (2000).

What is claimed is:

1. An isolated polypeptide comprising the amino acid sequence of SEQ ID NO: 2 and fragments thereof.

2. An isolated nucleic acid encoding the polypeptide of claim 1, and fragments thereof.

3. The isolated nucleic acid of claim 2, which comprises the nucleotide sequence of SEQ ID NO: 1.

4. The isolated nucleic acid of claim 3, wherein the fragments comprise the nucleotides 240 to 245 of SEQ ID NO: 1.

5. An expression vector comprising the nucleic acid of claim 2.

6. A host cell transformed with the expression vector of claim 5.

7. A method for producing the polypeptide of claim 1, which comprises the steps of:
(1) culturing the host cell of claim 6 under a condition suitable for the expression of the polypeptide; and
(2) recovering the polypeptide from the host cell culture.

8. An antibody specifically binding to the polypeptide of claim 1.

9. A method for diagnosing the diseases associated with the deficiency of KAP/Cdi1, in particular, lung cancer, in a mammal which comprises detecting the nucleic acid of any one of claims 2 to 4 or the polypeptide of claim 1.

10. The method of claim 9, wherein the lung cancer is small cell lung cancer.

11. The method of claim 9, wherein the detection of the nucleic acid of claim 2 comprising the steps of:
(1) extracting total RNA from a sample obtained from the mammal;
(2) amplifying the RNA by reverse transcriptase-polymerase chain reaction (RT-PCR) with a pair of primers to obtain a cDNA sample comprising the nucleotides 240 to 245 of SEQ ID NO: 1; and
(3) detecting whether the cDNA sample is obtained.

12. The method of claim 11, wherein one of the primers has a sequence comprising the nucleotides of SEQ ID NO: 1 containing nucleotides 240 to 245, and the other has a sequence complementary to the nucleotides of SEQ ID NO: 1 at any other locations downstream of nucleotide 245, or one of the primers has a sequence complementary to the nucleotides of SEQ ID NO: 1 containing nucleotides 240 to 245, and the other has a sequence comprising the nucleotides of SEQ ID NO: 1 at any other locations upstream of nucleotide 240.

13. The method of claim 11, wherein one of the primers has a sequence comprising the nucleotides of SEQ ID NO: 1 upstream of nucleotide 242, and the other has a sequence complementary to the nucleotides of SEQ ID NO: 1 downstream of nucleotide 243, or one of the primers has a sequence complementary to the nucleotides of SEQ ID NO: 1 upstream of nucleotide 242, and the other has a sequence comprising the nucleotides of SEQ ID NO: 1 downstream of nucleotide 243.

14. The method of claim 13, wherein the cDNA sample amplified from SEQ ID NO: 1 is 83 bp shorter than the cDNA sample amplified from KAP/Cdi1.

15. The method of claim 11 further comprising the step of detecting the amount of the amplified cDNA sample.

16. The method of claim 9, wherein the detection of the nucleic acid of any one of claims 2 to 4 comprises the steps of:
(1) extracting the total RNA of a sample obtained from the mammal;
(2) amplifying the RNA by reverse transcriptase-polymerase chain reaction (RT-PCR) to obtain a cDNA sample;
(3) bringing the cDNA sample into contact with the nucleic acid of any one of claims 2 to 4; and
(4) detecting whether the cDNA sample hybridizes with the is nucleic acid of any one of claims 2 to 4.

17. The method of claim 16 further comprising the step of detecting the amount of hybridized sample.

18. The method of claim 9, wherein the detection of the polypeptide of claim 1 comprises the steps of contacting the antibody of claim 8 with a protein sample obtained from the mammal, and detecting whether an antibody-polypeptide complex is formed.

19. The method of claim 18 further comprising the step of detecting the amount of the antibody-polypeptide complex.


SEQUENCE LISTING (2 sequences)

SEQ ID NO: 1 - DNA, 893 bp, Homo sapiens
  CDS (485)..(754)
tagaggccgagtcttcggccacccaaaggcggagtaagaaaccagaagcggatctgattggttgctggaagacgccgcgcccacctcacagaaggacgaaccagtgagctaagctgcggggcgcgggctcggccggggcaccggtgagtcgccggcgctgcagagggaggcggcactggtctcgacgtggggcggccagcgatggagccgcctatctttgtcacgagtgaattgttctcagtttctcggtttatgtgctcttccaggttgtaaatttaaagatgttagaagaaatgtccaaaaagatacagaagaactaaagagctgtggtatacaagacatatttgttttctgcaccagaggggaactgtcaaaatatagagtcccaaaccttctggatctctaccagcaatgtggaattatcacccatcatcatccaatcgcagatggagggactcctgacatagccagctgctgtgaaataatggaagagcttacaacctgccttaaaaattaccgaaaaaccttaatacactgctatggaggacttgggagatcttgtcttgtagctgcttgtctcctactatacctgtctgacacaatatcaccagagcaagccatagacagcctgcgagacctaagaggatccggggcaatacagaccatcaagcaatacaattatcttcatgagtttcgggacaaattagctgcacatctatcatcaagagattcacaatcaagatctgtatcaagataaaggaattcaaatagcatatatatgaccatgtctgaaatgtcagttctctagcataatttgtattgaaatgaaaccaccagtgttatcaacttgaatgtaaatgtacatgtgcagatattcctaaagttttattgac

SEQ ID NO: 2 - protein, 90 aa, Homo sapiens
MEELTTCLKNYRKTLIHCYGGLGRSCLVAACLLLYLSDTISPEQAIDSLRDLRGSGAIQTIKQYNYLHEFRDKLAAHLSSRDSQSRSVSR